(19) United States
(12) Patent Application Publication
(10) Pub. No.: US 2020/0093558 A1
(43) Pub. Date: Mar. 26, 2020

(54) SYSTEMS AND METHODS FOR DECONTAMINATED ENGAGEMENT OF VESSELS USING A DISPLACEABLE PLATE

(71) Applicant: Mikael Naygauz, Forrest Hills, NY (US)

(72) Inventor: Mikael Naygauz, Forrest Hills, NY (US)

(21) Appl. No.: 16/274,717

(22) Filed: Feb. 13, 2019

Related U.S. Application Data
(60) Provisional application No. 62/735,023, filed on Sep. 22, 2018.
(60) Provisional application No. 62/800,300, filed on Feb. 1, 2019.

(51) Int. Cl. A61B 50/30

(52) U.S. Cl. CPC A61B 50/30; A61B 2050/007

(57) ABSTRACT

The present invention relates, in some embodiments thereof, to systems, devices, and methods allowing for an engagement of two or more pharmaceutical vessels or pharmaceutical administration devices in a decontaminated manner. In some embodiments of the invention, the systems, devices and methods of the invention include a housing comprising a first compartment connectable to a first vessel's port; and a second compartment connectable to a second vessel's port, wherein the first and second compartments are engageable with each or integrally connected to each other, and wherein the first vessel is moveable within the housing from a first position wherein the vessel is connected to the first compartment, to a second position wherein the vessel is connected to the second compartment, wherein a substantially sterile aperture of the first vessel and/or second vessel is covered by a displaceable plate, and wherein prior to or at about the time of connection of the first vessel in the second position, the displaceable plate displaces from the aperture, allowing an substantially decontaminated engagement and fluid passageway between the first and second vessels.

### CROSS REFERENCE TO RELATED APPLICATIONS

This application claims the benefit of U.S. Provisional Application Nos. 62/735,023 filed Sep. 22, 2018 and 62/800,300 filed Feb. 1, 2019. The contents of these applications are all incorporated by reference as if fully set forth herein in their entirety.

### FIELD OF THE INVENTION

The present invention relates, in some embodiments thereof, to systems, devices, and methods allowing for an engagement of two or more pharmaceutical vessels or pharmaceutical devices in a decontaminated manner. In some embodiments of the invention, the systems, devices and methods of the invention allow for the air-free and sterile coupling of two or more pharmaceutical vessels or pharmaceutical administration devices.

### BACKGROUND OF THE INVENTION

The medicinal practice routinely involves administration of medical substances, such as, medicaments, fluids, nutritional substances and the alike, to patients or animals. The preparation and/or administration of such medicinal substances typically involves one or more transfers of those substances between pharmaceutical vessels (such as bags, bottles, containers, vials, etc.) or administration devices (such as, vials, syringes, infusion lines, connectors, etc.). Each such act of transferring substances between vessels or devices exposes the displaceable plates and/or surfaces of the vessels and/or administration devices and accordingly the medical substances themselves to contaminants present in ambient air or ambient air particles (e.g., bacteria, viruses, funguses, spores, pyrogens, dirt). In addition, displaceable plates and/or vessel/device surfaces are further prone to contamination due to physical contact of the interfaces/surfaces, for example, with nonsterile gloves, or devices.

Such contaminations are a major problem in the healthcare setting since contaminants, once invading within medicinal substances, may pose substantial danger if administered intracorporeally to patients.

Typical displaceable plates/vessel surfaces/device surfaces of pharmaceutical vessels or pharmaceutical administration devices include rubber bungs and/or stoppers covered by a cap and/or seal that can be flicked off and/or are removed prior to usage thereof. These rubber bungs/stoppers are used to allow penetration by a needle attached to a syringe or by other medical connectors having piercing members such as spikes. When the cap and/or seal is flicked off and/or removed, the rubber bung and/or stopper is exposed to ambient air and to contaminants present therein. Accordingly, exposure of displaceable plates/vessel surfaces/device surfaces to ambient air may involve contamination of the interfaces and consequently contamination of a beneficial substance to be provided to a patient.

Existing systems include U.S. Pat. Nos. D720,067; D717,947; D703,812; D690,418; D639,939; D637,713; 9,790,011; 9,775,777; 9,561,326; 9,493,281; 9,492,353; 9,309,020; 9,173,816; 9,168,203; 9,162,803; 9,039,672; 8,926,583; 8,827,978; 8,790,330; 8,662,985; 8,657,803; 8,622,985; 8,562,583; 8,545,475; 8,523,838; 8,491,563; 8,480,646; 8,449,521; 8,381,776; 8,336,587; 8,328,772; 8,287,513; 8,225,826; 8,075,550; 8,029,747; 7,998,134; 7,975,733; 7,942,860; 7,867,215; 7,744,581; 7,731,678; 7,387,216; 7,306,584; 6,875,203; 6,729,370; 6,715,520; 6,602,239; 6,409,708; 6,343,629; 6,162,199; 6,113,583; 6,063,068; 5,893,397; 5,876,380; 5,832,971; 5,807,374; 5,746,733; 5,569,235; 5,462,535; 5,405,326; 5,292,318; 5,279,582; 4,944,723; 4,932,947; 4,932,937; 4,919,657; 4,915,701; 4,826,489; 4,673,404; 4,564,054; 3,610,241; 3,605,743; 3,587,575; 3,583,399; 3,578,037; 3,556,099; 3,552,387; 3,406,686; 3,380,450; 3,375,825; 3,342,180; 3,330,282; 3,330,281; 3,306,290; 3,255,752; 3,253,592; 3,076,456; 2,972,991; 2,922,419; US20160262982; US20160038373; US20150209568; US20140183196; US20140016570; US20140007973; US20140000754; US20130184672; US20130006200; US20120209238; US20120209218; US20120203194; US20110284561; US20110186177; US20110125128; US20110108158; US20110098647; US20100249745; US20100198182; US20100152669; US20100147402; US20100036319; US20100004602; US20090057258; US20080312634; US20080223484; US20080171981; US20060276759; US20050215976; US20030199847; US20030187420; US20020130100; US20020115981; US20020099354; ES2577377T3; EP2852367B1; EP2666513; EP2155141B1.

In order to overcome this obstacle, the current medical practice involves swabbing the surface of a displaceable plate/vessel surface/device surface with a disinfecting agent, such as 70% isopropyl alcohol, prior to accessing/piercing the displaceable plate/vessel surface/device surface. Other methods include i.v. (intravenous) rooms which are used for the sterile preparation of i.v. medications. Such rooms, to keep medicinal preparations as sterile as possible, are equipped with special instruments including, hoods with air filtration systems (e.g., HEPA filters), ventilation systems and air pressure systems. Additionally, those rooms necessitate that the medical staff working in these rooms are properly garmented, are properly trained, and require aseptic techniques, and employ quality control and validation processes. These systems require regular upkeep by certified personnel and require regular cleaning. These systems are therefore expensive, labor intensive, and require regular maintenance and testing to assure that they are operating effectively. The above described systems and methods are either cumbersome and expensive or inefficient in addressing the problem of reducing/eliminating contaminants on displaceable plates.

Thus, there is a long felt and unmet need for pharmaceutical vessels, devices, systems and/or methods that afford transfer of medical substances in a sterile manner. There is a need for reliable, user friendly and cost-effective solutions allowing contaminant-free engagement of vessels and devices for drug preparation and administration processes.

### SUMMARY OF THE INVENTION

Objects of the invention are achieved by providing systems, devices, connectors and methods for engaging medical devices, such as vessels, in a decontaminated manner.

Objects of the invention are achieved by providing systems, containers, devices, vessels and methods which are directed to transferring medical substances in an efficient, user-friendly and sterile manner.

The present invention is based on a container, device, system or connector that comprises a housing with two or more compartments that are connectable or integrally attached to each other. Each of the compartments configured to connect a vessel and allow movement of the vessel(s) along a path between the compartments, thereby establishing a fluid communication between the vessels. The one or more vessels include a displaceable plate that seals a sterile aperture of the vessels and configured to displace from the aperture and thereby allow a substantially sterile communication between the sustainably sterile apertures of the vessels.

In an aspect of the invention, the present invention provides a system for a decontaminated engagement of vessels, the system comprises:

a housing comprising a first compartment connectable to a first vessel's port; and a second compartment connectable to a second vessel's port,

wherein the first and second compartments are engageable with each or integrally connected to each other, and wherein the first vessel is moveable within the housing from a first position wherein the vessel is connected to the first compartment, to a second position wherein the vessel is connected to the second compartment, wherein a substantially sterile aperture of the first vessel and/or second vessel is covered by a displaceable plate, and wherein prior to or at about the time of connection of the first vessel in the second position, the displaceable plate displaces from the aperture, allowing an substantially decontaminated engagement and fluid passageway between the first and second vessels.

In one or more embodiments, the first compartment is provided when attached to a port of the first vessel. In one or more embodiments, the first and second compartments are engageable with each other via an engagement mechanism. In one or more embodiments, the engagement mechanism is selected from a thread, a luer, an adhesive, a clamping rail, a teeth and complementary dents, a clamping arm and a ratchet teeth mechanism. In one or more embodiments, the first and second compartments are horizontally engageable with each allowing an axial movement of the first vessel within the housing. In one or more embodiments, the second position of the first vessel comprises a substantially vertical alignment between the first and second vessels.

In one or more embodiments, the displaceable plate displaces externally. In one or more embodiments, the displaceable plate is a film, a plastic, a metal alloy, or a combination thereof.

In one or more embodiments, the movement of the first vessel within the housing is via a sliding mechanism. In one or more embodiments, the sliding mechanism includes a rail. In one or more embodiments, the sliding mechanism disposed on the first vessel, second vessel, first compartment and/or second compartment. In one or more embodiments, the first vessel and the second vessel each include a displaceable plate and wherein the displaceable plates externally displace about the same time, or consecutively. In one or more embodiments, the displaceable plate displaces from the aperture at about the time of movement of the first vessel within the housing.

In one or more embodiments, the system further comprising a tab attached to the displaceable plate, the tab configured to allow for the external displacement of the plate. In one or more embodiments, the tab is configured to be pulled axially by a user, the axial pulling of the tab configured to displace the plate.

In one or more embodiments, the system further comprising a hinge mechanism connecting the plate to a vessel and configured to provide the external displacement of the plate.

In one or more embodiments, the system allows a substantially air particle-free engagement between the first vessel and the second vessel, wherein air particles interfacing the displaceable plate are captured between the plates.

In one or more embodiments, an audible sound is emitted at or about the time of engagement between the first compartment and the second compartment, displacement of the displaceable plate, engagement between the first and second vessels, movement of the first vessel in the housing and/or formation of a fluid passageway between the vessels.

In one or more embodiments, the system further comprising a locking-unlocking mechanism between the first compartment and the second compartment, the locking mechanism configured to lock the compartments to each other.

In one or more embodiments, the system further comprising a pressing mechanism on at least one of the first compartment and second compartment, the pressing mechanism configured to allow a tight connection between the compartments. In one or more embodiments, the pressing mechanism includes a spring that presses the first compartment against a wall of the second compartment.

In one or more embodiments, the displaceable plate is configured to seal and/or cover an aperture of the vessel.

In one or more embodiments, the decontamination system results in air particle-free engagement of the first vessel and the second vessel.

In one or more embodiments, air particles are entrapped between the compartments upon the engagement of the compartments. In one or more embodiments, air particles (contaminants) are entrapped between the first displaceable plate and the second displaceable plate at about the time of engagement of the first displaceable plate to the second displaceable plate.

In one or more embodiments, the system further comprises an engagement mechanism configured to provide a secure engagement between the first compartment and the second compartment. In one or more embodiments, the engagement mechanism is located on/within the first compartment and/or on/within the second compartment.

In one or more embodiments, the system further comprises an engagement mechanism configured to provide a secure engagement between the first vessel and the second vessel. In one or more embodiments, the engagement mechanism is located on the first vessel and/or on the second vessel.

In one or more embodiments, the engagement mechanism is selected from a thread, a luer, an adhesive, and a ratchet teeth mechanism. Optionally, a twisting, turning, and/or snapping motion is associated with the engagement. In an exemplary embodiment, a twisting motion is associated with a thread-luer mechanism. In an exemplary embodiment, a snapping motion is associated with a ratchet teeth mechanism. In one or more embodiments, when the first vessel and the second vessel engage via the engagement mechanism, an airtight seal is formed between the first vessel and the second vessel. In one or more embodiments, when the first vessel and the second vessel engage via the engagement mechanism a sealed aperture is formed between the first vessel and the second vessel. In one or more embodiments, when the first vessel engages the first compartment, an airtight seal is formed between the first vessel and the first compartment. In one or more embodiments, when the first vessel engages the first compartment, a hermetic coupling is established between the first vessel and the first compartment.

In one or more embodiments, the system further comprises a sliding mechanism on the first vessel and/or second vessel. In one or more embodiments, the system further comprises a sliding mechanism on the first compartment and/or second compartment. In one or more embodiments, the sliding mechanism configured to provide external displacement of the displaceable plate. In one or more embodiments, the sliding mechanism configured to provide displacement of the displaceable plate from the first vessel or the second vessel. In one or more embodiments, the sliding mechanism configured to provide displacement of the displaceable plate from the first vessel and the second vessel. In one or more embodiments, the sliding mechanism configured to provide movement of a vessel within the housing. In one or more embodiments, the sliding mechanism configured to provide movement of a vessel's port within the housing. In one or more embodiments, the sliding mechanism configured to provide movement of a portion of a vessel's port within the housing. In one or more embodiments, the sliding mechanism configured to provide movement of a vessel along the housing. In one or more embodiments, the sliding mechanism configured to provide movement of a vessel's port along the housing. In one or more embodiments, the sliding mechanism configured to provide movement of a portion of a vessel's port along the housing. In one or more embodiments, the sliding mechanism configured to provided movement of a first vessel along a second compartment of the housing. In one or more embodiments, the sliding mechanism configured to provide movement of a vessel's port along a second compartment of a housing. In one or more embodiments, the sliding mechanism is a rail mechanism. In one or more embodiments, the sliding mechanism is a rail. In one or more embodiments, the rail mechanism is disposed on at least one of the first compartment and the second compartment. In one or more embodiments, the rail is disposed along at least one of the first vessel and the second vessel. In one or more embodiments, the vessel is a container. In one or more embodiments, the vessel is a connector. In one or more embodiments, the vessel is a syringe.

In one or more embodiments, the system comprises a seal or a cover disposed between a vessel and a displaceable plate. In one or more embodiments, the seal or cover is frangible. In one or more embodiments, the seal or cover is pierceable. In one or more embodiments, the seal is rupturable. In one or more embodiments, the cover is rupturable. In one or more embodiments the seal is sterile. In one or more embodiments, the cover is sterile. In one or more embodiments, the seal is not exposed to air. In one or more embodiments, the cover is not exposed to air. In one or more embodiments, the vessel is a container. In one or more embodiments, the vessel is a connector. In one or more embodiments, the vessel is a syringe.

In one or more embodiments, the system comprises a first seal or a first cover disposed between a first vessel and a first displaceable plate and a second seal or a second cover disposed between a second vessel and a second displaceable plate. In one or more embodiments, the first seal or first cover and a second seal or a second cover is frangible. In one or more embodiments, the first seal or first cover and a second seal or a second cover is frangible.

In one or more embodiments, the system further comprises a piercing member on the first and/or second vessel or on the first and/or second compartment, the piercing member configured to pierce a sealed aperture of the first and/or second vessel, thereby allowing fluid to pass between the vessels. In one or more embodiments, the piercing member is a needle. In one or more embodiments, the needle is a hollowed needle.

In one or more embodiments, the first and second displaceable plates are externally displaced via a sliding and/or a pulling motion. In one or more embodiments, the first and second displaceable plates are displaced via a sliding and/or a pulling motion. In one or more embodiments the first displaceable plate is externally displaced via a sliding and/or a pulling motion. In one or more embodiments, the first displaceable plate is displaced via a sliding and/or a pulling motion. In one or more embodiments the second displaceable plate is externally displaced via a sliding and/or a pulling motion. In one or more embodiments the second displaceable plate is displaced via a sliding and/or a pulling motion. In one or more embodiments, the first displaceable plate is externally displaced while the second displaceable plate is not externally displaced. In one or more embodiments, the first displaceable plate is displaced while the second displaceable plate is not displaced. In one or more embodiments, the second displaceable plate is externally displaced while the first displaceable plate is not externally displaced. In one or more embodiments, the second displaceable plate is displaced while the first displaceable plate is not displaced. In one or more embodiments the first displaceable plate is displaced via a sliding, pulling, and/or pushing motion while the second displaceable plate is not displaced. In one or more embodiments, the second displaceable plate is displaced via a sliding, pulling, and/or pushing motion while the first displaceable plate is not displaced. In one or more embodiments, the first and second displaceable plates are both displaced via a sliding, pulling, and/or pushing motion. In one or more embodiments, the first and second displaceable plates are both externally displaced via a sliding, pulling, and/or pushing motion.

In one or more embodiments, when the first displaceable plate and the second displaceable plate are displaced, the connection between the first vessel and the second vessel is decontaminated. In one or more embodiments, when the first and/or second displaceable plate slides off the first and/or second sliding mechanism, the hermetic connection between the first vessel and the second vessel is maintained. In one or more embodiments, when at least one of the first displaceable plate and the second displaceable plate are displaced, a sterile coupling between the first vessel and the second vessel is established.

In one or more embodiments, the displaceable plate externally displaces from the vessel's aperture through a slit or the aperture or port of the vessel. In one or more embodiments, the displaceable plate displaces between ratchet teeth. In one or more embodiments, the displaceable plate displaces between two ratchet teeth of the first compartment.

In one or more embodiments, the first vessel and/or the second vessel are selected from a vial, a bag, a bottle, a syringe, an infusion line, a connector, a filter, a manifold, a container port, a bag port, a bottle port, a vial port, and combinations thereof.

In one or more embodiments, circumferential ends of at least one of or both of the first and second displaceable plate ends are concealed within the respective compartment. In one or more embodiments, the first displaceable plate is concealed within the first compartment at about the time and/or after engagement of the first vessel to the first compartment of the housing. In one or more embodiments, the second displaceable plate is concealed within the second compartment. In one or more embodiments, the second displaceable plate is entirely concealed within the second compartment of the second vessel. In one or more embodiments, the second displaceable plate is partially concealed within the second compartment. In one or more embodiments, a portion of the second displaceable plate is concealed within the second compartment and a portion of the second displaceable plate is exposed to the first compartment. In one or more embodiments, a portion of the second displaceable plate is concealed within the second compartment and a portion of the second displaceable plate is exposed to air. In one or more embodiments, the air is ambient air.

In one or more embodiments, the first vessel and the second vessel are in fluid communication with one another after external displacement of the displaceable plate(s) and/or movement of the first vessel from the first position to the second position.

In one or more embodiments, the first displaceable plate is coupled to the first vessel and the second displaceable plate is coupled to the second vessel.

In a further aspect, the present invention provides a system for the engagement of vessels, comprising:
providing a first vessel having a first displaceable plate, the first displaceable plate having an engagement mechanism configured to engage a second displaceable plate; and
providing a second vessel having a second displaceable plate, the second displaceable plate having an engagement mechanism configured to engage the first displaceable plate.

In one or more embodiments, the engagement mechanism of the first displaceable plate is at least one tooth and the engagement mechanism of the second displaceable plate is at least one dent. In one or more embodiments, the engagement mechanism of the first displaceable plate are teeth and the engagement mechanism of the second displaceable plate are dents.

In one or more embodiments, the first vessel is a container and the second vessel is a container. In one or more embodiments, the first vessel is a bag and the second vessel is a bottle. In one or more embodiments, the first vessel is a bag and the second vessel is a vial. In one or more embodiments, the first vessel is a bottle and the second vessel is a vial. In one or more embodiments, the first vessel is a container and the second vessel is a syringe. In one or more embodiments, the first vessel is a container and the second vessel is a syringe.

In a further aspect, the present invention provides a container having a displaceable plate, the displaceable plate having an engagement mechanism configured to engage a second displaceable plate.

In one or more embodiments, the container further comprising a rail mechanism configured to allow the displaceable plate to travel along the rail mechanism.

In a further aspect, the present invention provides a method of allowing a decontaminated engagement between vessels, the method comprising the steps of:

providing a first compartment and a second compartment;

engaging a first vessel's port to the first compartment and/or engaging a second vessel's port to the second compartment, wherein an aperture of the first and/or second vessels is sealed by a displaceable plate;

moving the first vessel along the compartments from a first position wherein the vessel is connected to the first compartment, to a second position wherein the vessel is connected to the second compartment; and externally displacing the displaceable plate(s), thereby allowing a substantially decontaminated engagement and fluid communication between the vessels.

In one or more embodiments, the step of externally displacing the displaceable plates occurs via a sliding motion. In one or more embodiments, the displacement of the displaceable plate is via a rail mechanism. In one or more embodiments, the step of externally displacing the displaceable plate occurs via a peeling motion.

In one or more embodiments, the first vessel and second vessel each include a displaceable plate and step of externally displacing the displaceable plates occurs at about the same time, or consecutively. In one or more embodiments, the displaceable plate(s) displace(s) from the aperture at about the time of movement of the first vessel within the housing.

In one or more embodiments, the method further comprising connecting the first compartment to the second compartment. In one or more embodiments, connecting the first compartment to the second compartment is via an engagement mechanism. In one or more embodiments, connecting of the first compartment to the second compartment occurs prior to externally displacing the displaceable plate(s). In one or more embodiments, connecting the first compartment to the second compartment includes locking of the first compartment to the second compartment. In one or more embodiments, connecting of the first compartment to the second compartment is selected from a twisting motion, a pushing motion, a sliding motion, a turning motion, or combinations thereof.

In one or more embodiments, the method further comprising connecting the first displaceable plate to the second displaceable plate. In one or more embodiments, connecting the first displaceable plate to the second displaceable plate is via an engagement mechanism. In one or more embodiments, connecting the first displaceable plate to the second displaceable plate occurs prior to externally displacing the displaceable plate(s). In one or more embodiments, connecting the first displaceable plate to the second displaceable plate includes locking of the first displaceable plate to the second displaceable plate. In one or more embodiments, connecting the first displaceable plate to the second displaceable plate is selected from a twisting motion and a turning motion. In one or more embodiments, connecting the first displaceable plate to the second displaceable plate is via a pushing motion. In one or more embodiments, connecting the first displaceable plate to the second displaceable plate is via a pulling motion.

In one or more embodiments, the method further comprising piercing a seal or a cover of the first and/or second vessel(s). In one or more embodiments, piercing the seal or cover occurs when the first vessel disposed in the second position.

In one or more embodiments, moving the first vessel is via an axial horizontal motion. In one or more embodiments, moving the first vessel along the compartments allows a substantially vertical alignment between the first vessel and the second vessel. In one or more embodiments, the step of moving the first vessel occurs via a sliding motion.

In one or more embodiments, the step of externally displacing the first displaceable plate and the second displaceable plate occurs via sliding the first displaceable plate and the second displaceable plate axially.

In one or more embodiments, the external displacement of the first displaceable plate and the second displaceable plate is in the same direction. In one or more embodiments, the external displacement of the first displaceable plate and the second displaceable plate is to opposite directions.

In one or more embodiments, the displacement of the first displaceable plate and the second displaceable plate allows a decontaminated fluid passageway between the vessels via the sterile apertures of the vessels. In one or more embodiments, the displacement of the first displaceable plate and the second displaceable plate allows a decontaminated engagement between the vessels via the sterile apertures of the vessels.

In one or more embodiments, the method further comprises connecting the first displaceable plate to the second displaceable plate via an engagement mechanism.

In one or more embodiments, connecting of the first displaceable plate to the second displaceable plate occurs prior to externally displacing the first and second displaceable plates. In one or more embodiments, the connecting of the first displaceable plate to the second displaceable plate is selected from a twisting of a thread onto a complementary thread, attaching ratchet teeth to a complementary retention member, and adhering a first displaceable plate to the second displaceable plate via an adhesive.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1E are schematic front view illustrations presenting an exemplary decontamination system which comprises a housing with a first compartment and a second compartment, the first compartment configured to attach a first vessel via ratchet teeth and the second compartment provided when attached to a second vessel, the vessels configured to engage with each other directly or via the second compartment of the housing and to fluidly communicate with each other following an external displacement of at least one displaceable plate, according to some embodiments of the invention.

FIG. 2 is a side view illustration of the system of FIGS. 1A-1E, according to some embodiments of the invention.

FIGS. 3A-3D are schematic front view illustrations presenting an exemplary decontamination system wherein the first compartment configured to attach a first vessel via a thread mechanism, according to some embodiments of the invention.

FIGS. 4A-4D are schematic front view illustrations presenting an exemplary decontamination system wherein the first compartment configured to attach a first vessel via a clamping rail mechanism, according to some embodiments of the invention.

FIGS. 5A-5E are schematic front view illustrations presenting an exemplary decontamination system wherein the first compartment provided when attached to a port of the first vessel and configured to attach a second compartment via teeth and complementary dents, according to some embodiments of the invention.

FIGS. 6A-6D are schematic front view illustrations presenting an exemplary decontamination system wherein the first compartment provided when attached to a port of the first vessel and configured to attach a second compartment via a pressing mechanism and a lock-unlock mechanism, according to some embodiments of the invention.

FIGS. 7A-7D are bottom view isometric illustrations presenting an exemplary decontamination system wherein the first compartment and second compartments integrally attached within a housing, the system comprises a pressing mechanism for the attachment of a first vessel to the first compartment of the housing, according to some embodiments of the invention.

It should be appreciated that for simplicity and clarity of illustration, members shown in the figures have not necessarily been drawn to scale. For example, the dimensions of some of the members are exaggerated relative to each other for clarity. Further, where considered appropriate, reference numerals have been repeated among the figures to indicate corresponding members.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary medical containers/devices for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The present invention relates to a decontamination device, system, and/or connector and to a method of use thereof. More particularly, the present invention relates to a decontamination device for preparing and/or administering a beneficial substance to a patient.

The decontamination device and/or housing and/or connector may be coupled to and/or integral with one or more vessels. The decontamination device and/or housing and/or connector may form a unitary structure with a vessel.

The device, system and/or connector of the invention inter alia affords the following attributes:

removes ambient air particles and air contaminates from the interface between vessels;

allows a substantially decontaminated fluidic passageway between vessels;

provides a low-cost portable and optionally disposable air purging solutions for fluid transfer, preparation and/or administration.

The present invention relates to devices, methods and systems allowing an engagement of medical containers or devices in a sterile manner. The present invention relates to devices, methods and systems allowing fluid passageway between medical containers or devices in a sterile manner. The invention provides a solution to an unmet and long felt need in the medical setting and allows connecting in a substantially sterile manner, two or more vessels. The herein disclosed devices and systems are user friendly, cost effective and abolish the need for complicated and expensive known methods for transferring medical substances. In an embodiment of the invention, the herein disclosed devices and systems are disposable. In an embodiment of the invention, the herein disclosed devices and systems are non-disposable. In an embodiment of the invention, the herein disclosed vessels and devices are intended for one-time use. In an embodiment of the invention, the herein disclosed vessels and devices are intended to be reused.

In an aspect of the invention, the devices and systems comprise a housing that includes a first compartment and a second compartment which can engage with each other or which are provided when attached already, optionally demonstrate an integral structure. The housing allows connection of two or more vessels. A first vessel, a port of a first vessel, and/or a portion of a first vessel is moveable within and/or along the housing and may move from a first position in which the vessel is attached to the first compartment to a second position in which the vessel is attached to second compartment and/or to a second vessel. At least one of the vessels includes a displaceable plate which covers an aperture of the vessel. Such movement of the first vessel allows a fluid communication between the vessels and/or displacement of the displaceable plates. In one or more embodiments, the vessels are connected to each other directly. In one or more embodiments, the vessels are connected to each other indirectly, via the housing or compartment of the system/device. In one or more embodiments, the housing includes two or more, or three or more or five or move compartments. Optionally, the compartments are disposed next or adjacent each other, providing a left compartment and a right compartment. Optionally, the chambers are disposed one above the other, providing a top chamber and a bottom chamber. In one or more embodiments, the housing is made of a plastic material. In one or more embodiments, the plastic material is a rigid plastic material. In one or more embodiments, the housing is made of a metal alloy, a glass material, a plastic material and combinations thereof.

When the displaceable plate(s) displace a substantially sterile engagement and/or fluid passageway between vessels is provided.

Accordingly, the herein disclosed invention allows transferring medical substances in a contaminants-free, or in a substantially contaminants-free manner. In one or more embodiments, the herein disclosed invention allows connecting vessels in a contaminants-free, or in a substantially contaminants-free manner. In one or more embodiments, the herein disclosed invention allows connecting vessels in a sterile manner. In one or more embodiments, the herein disclosed invention allows connecting vessels while eliminating ambient air and/or ambient air particles from between the site of vessel engagement/coupling.

In one or more embodiments, the herein disclosed invention provides a fluidic passageway or communication between medical containers in a contaminants-free, or in a substantially contaminants-free manner.

In one or more embodiments, the herein disclosed invention, allows to substantially decrease the chances to introduce a contamination within a medical substance, when preparing or administering medical substances.

As used herein, the term “substances” refers to various types of materials that should be kept sterile. The substances may be liquid, solid, semi-solid, or gas. In one or more embodiments, the substances are “medical substances”. As used herein the term “medical substances” refers to and encompasses any of the various medical drugs, fluids, nutritional products, solid powders, suspensions, liquids solutions and the like.

As used herein, the term “contaminant-free” is interchangeable with the term “sterile”, “disinfected”, and “decontaminated”. The term refers to substances that are free or substantially free of ambient air particles and/or pathogens, including but not limited to dirt, bacteria, viruses, funguses, spores, and/or pyrogens. Typically, when less or no air is introduced within medical substances, the chances of contamination by pathogens, such as, bacteria, viruses, funguses, spores, pyrogens or the alike is completely abolished or significantly reduced.

As used herein, “substantially contaminants-free” means significantly less ambient air present when transferring medical substances with the herein disclosed vessels and systems, as compared to comparable conditions for transferring medical substances without the herein disclosed vessels and systems.

As used herein, the term “ambient air particles” is interchangeable with the term “environmental air particles” and refers to air particles present in a non-filtered or filtered environment. For example, air can be purified by filters, such as a High Efficiency Particulate Air (HEPA) filter.

As used herein, the term “displaceable plate” encompasses any surface, layer plane, wall, or the alike that can be attached to a vessel. Optionally, the term encompasses a structure that can be coupled to a vessel and that can engage with a complementary displaceable plate located on another vessel. In an embodiment of the invention, the displaceable plate is disposed to seal an aperture of a vessel. In an embodiment of the invention, the displaceable plate is disposed within an aperture of a compartment. In an embodiment of the invention, the displaceable plate may seal or cover an aperture of a vessel. In an embodiment of the invention, the displaceable plate may seal or cover an opening for fluid passageway of a vessel. In an embodiment of the invention, the displaceable plate may be a surface of a container. In an embodiment of the invention, the displaceable plate may be a surface of a device. In an embodiment of the invention, the displaceable plate may be a surface of a vessel. In an embodiment of the invention, the displaceable plate abuts a container. In an embodiment of the invention, the displaceable plate is surface mounted to a container. In an embodiment of the invention, the displaceable plate is surface mounted to a wall of the container. In an embodiment of the invention, the displaceable plate is flush mounted to a container.

In an embodiment of the invention, the displaceable plate abuts a device. In an embodiment of the invention, the displaceable plate is surface mounted to a device. In an embodiment of the invention, the displaceable plate is surface mounted to a wall of a device. In an embodiment of the invention, the displaceable plate is flush mounted to a device.

In an embodiment of the invention, the displaceable plate abuts a vessel. In an embodiment of the invention, the displaceable plate abuts a wall of a vessel. In an embodiment of the invention, the displaceable plate is surface mounted to a vessel. In an embodiment of the invention, the displaceable plate is surface mounted to a wall of a vessel. In an embodiment of the invention the displaceable plate is flush mounted to a vessel. In an embodiment of the invention, the displaceable plate is flush mounted to a wall of a vessel. In one or more embodiments, a plurality of displaceable plates abuts a vessel and/or device. In an embodiment of the invention, a plurality of displaceable plates is surface mounted to a vessel. In an embodiment of the invention, a plurality of displaceable plates is surface mounted to a at least one wall of a vessel. In an embodiment of the invention, a plurality of displaceable plates is surface mounted to a vessel. In an embodiment of the invention, a plurality of displaceable plates is flush mounted to a vessel. In an embodiment of the invention, a plurality of displaceable plate is flush mounted to at least one wall of a vessel. In one or more embodiments, a plurality of displaceable plates is attached to a vessel, a container, and/or a device.

In an embodiment of the invention, the present invention discloses a first displaceable plate and a second displaceable plate. The first displaceable plate and the second displaceable plate may be similar or may be different in size and/or shape. In one or more embodiments, the exterior surface area facing ambient air, of the displaceable plates may be equal. In one or more embodiments, the exterior surface area facing ambient air, of the displaceable plates may be non-equal. In one or more embodiments, the difference in surface area between the exterior surface area of a plurality of displaceable plates is less than 0.3 inches squared. For example, less than 0.2 inches squared, or less than 0.1 inches squared.

In one or more embodiments, the displaceable plate has a thin film layer. In an exemplary embodiment, the displaceable plate may present a thickness of about 1 mm or below. Alternatively, the displaceable plate may present a thickness of above 1 mm. The displaceable plate may be manufactured from various materials, including, an elastic polymer. The displaceable plate may be made from a plastic material. The plastic material may be rigid. The displaceable plate may be a rubber or a polymer. The displaceable plate may be formed of a metal material (e.g., a metal alloy). In yet a further exemplary embodiment, the displaceable plate may preset the form of a thin film made of an aluminum foil, cling wrap, or the alike. The metal alloy may include, for example, aluminum. In yet a further exemplary embodiment, the displaceable plate may preset the form of a thin film made of an aluminum foil, or the alike. In one or more embodiments, the displaceable plate is attached to, or is integrally formed with a vessel, or a port thereof. In one or more embodiments, the displaceable plate is peelable from the vessels. In one or more embodiments, the peelable displaceable plate is attached to a vessel via an adhesive. In one or more embodiments, the displaceable plate is attached to a vessel via a frangible seal. In one or more embodiments, the displaceable plate is attached to a vessel via a rupturable seal. In one or more embodiments, the peelable displaceable plate may be detached from a vessel surface by the force of a user's hand. In one or more embodiments, the displaceable plate is movable and/or slidable along a rail located along a length of the aperture of a vessel. In one or more embodiments, the displaceable plate is movable or slidable along a rail located along a length of the first and/or second housing, and/or vessel's compartment. In one or more embodiments, the displaceable plate may be covered by a cover (not shown). In one or more embodiments, the cover may be a peelable cover. In one or more embodiments, a sterilizing or disinfecting substance may be applied onto the displaceable plate and the cover. In one or more embodiments, the sterilizing or disinfecting substance may be an alcohol. In one or more embodiments, the alcohol may be 70% isopropyl alcohol.

The displaceable plates may be coupled within any portion of the vessel. For example, the displaceable plates may be connected onto the vessels' body or aperture, the vessels' wall or port thereof.

In one or more embodiments, the displaceable plate is configured to prevent contaminants from entering the fluid passageway between a first vessel and a second vessel. In one or more embodiments, the displaceable plate is configured to prevent contaminants from entering the interior of the vessel's body. In one or more embodiments, the displaceable plate is configured to prevent contaminants from entering a fluid housed inside the vessel's body. In one or more embodiments, the displaceable plate is configured to prevent contaminants from contacting an internal compartment of the vessel.

In one or more embodiments, the displaceable plate is displaceable and/or moveable. As used herein the term “displacement” refers to a displacement (i.e., dislocation) of the herein disclosed displaceable plate. In an embodiment of the invention, the displacement is external, namely, outside the vessels being connected by the herein disclosed system/device/container/connector. In an embodiment of the invention, the displacement is external to the fluid communication established following (or at about the time of) engagement of the vessels being connected by the herein disclosed system, methods and devices. In an embodiment of the invention, the displacement maintains a hermetic seal of the displaceable plates and/or the vessels. The displacement may occur via a sliding motion, or by a pulling out motion, or by a pushing motion, or by peeling the displaceable plate(s). In an embodiment of the invention, the displacement occurs for one of the vessels. In an embodiment of the invention, the displacement occurs for both vessels. The external displacement may optionally occur simultaneously for both displaceable plates or may occur consecutively. In an embodiment of the invention, the external displacement establishes a fluid passageway between two or more vessels. In one or more embodiments, at about the time of, prior to, or after external displacement of the displaceable plate, the aperture at which the displaceable plate resides reseals, allowing a hermetic airtight connection between two or more vessels.

As used herein the term “vessel” refers to any device utilized for containing, housing or transferring substances as herein disclosed. In one or more embodiments, the vessel may be used for containing medical substances. In an embodiment of the invention, the vessel is a medical vessel. In an embodiment of the invention, the vessel is a medical device. In an embodiment of the invention, the vessels are used for, and adapted to allow connection to another vessel(s). In an embodiment of the invention, the vessel may be a medical container utilized for accommodating medical substances. Various types of medical containers are contemplated. The medical container may be selected, without limitation, from a vial, a bag, a chamber, a housing, a bottle, and the alike. In an embodiment of the invention, the term vessel further encompasses members that can be used to connect between vessels. In accordance with this embodiment, the vessel may be selected, without limitation, from a connector, a port, a syringe, an infusion line, a tubing, a syringe, a filter, a spike, a port and a manifold. Optionally, a vessel is provided when integrally attached to a device or a system or a connector as herein disclosed. Alternatively, additionally or optionally, a vessel is provided when disconnected from, and can be coupled to the device or system or connector as herein disclosed.

In one or more embodiments, the system/housing/device optionally includes one or more safety mechanisms to secure one or more components of the system/device/housing and/or ensure proper use thereof. In one or more embodiments, the system/device/housing includes at least one safety mechanism selected from a valve safety mechanism, a piercing member safety mechanism, a cap safety mechanism, a rail safety mechanism, and a detachment safety mechanism. In one or more embodiments, the housing includes at least one safety mechanism, to secure at least one component of the housing, selected from: a valve, a one-way valve, a rail, a displaceable plate, a piercing member, a vessel, and a combination thereof.

Various forms of the safety mechanisms are herein contemplated. For example, the safety mechanism may have a stopper, a lock structure or a brake structure. In an exemplary embodiment, the safety mechanism includes, without limitation, a plunger, a latch, a knob, a pin, a shaft, or a combination thereof. The safety mechanism may include an unlocking member, such as a cable which is connected to the safety mechanism and when retracted or fastened releases the safety mechanism. Optionally, the safety mechanism is hingedly connected to an inner wall or compartment of the housing. Alternatively, the safety mechanism is connected to the housing from an external surface thereof. Optionally, the safety mechanism is rotatable within the housing. The safety mechanism may rotate about an angle of up to 360 degrees within the housing. For example, the safety mechanism may rotate about an angle of up to 180, or 90 degrees within the housing. Optionally, the safety mechanism is movable from a retracted position wherein it is concealed within an inner wall or compartment of the housing to an extended position wherein it protrudes from an inner wall of the housing. Optionally, the movement between an extended position and a retracted position of the safety mechanism is mediated by a spring which is optionally attached to the safety mechanism, optionally from below or from the side. The safety mechanism may be manufactured from various rigid materials, such as, a plastic or a metal (e.g., a metal alloy). Alternatively, or additionally, the safety mechanism may be manufactured from various semi-rigid or resilient materials.

The safety mechanisms may be optionally unlocked or locked by an engagement of the vessel with the housing, manually by a user pressing a dedicated button, by movement of the vessel within the housing, and/or by detachment of the vessel from the housing. Optionally, the vessel may comprise an unlocking member that engages the safety mechanism and unlocks the safety mechanism. Further optionally, or additionally the vessel may comprise a locking member that engages the safety mechanism and locks the safety mechanism with the vessel, or with a compartment or a component within the housing. Such locking or unlocking member may be disposed on the vessel's surface. In one or more embodiments, the safety mechanism is unlocked by and/or locked to the vessel via a motion selected from pushing, pulling (slight pulling), twisting, turning, sliding, push and twist, and/or twist and push, or a combination thereof.

In one or more embodiments, the safety mechanism makes an audible sound upon use thereof (e.g., when unlocked/released). In an exemplary embodiment the audible sound may be a “CLICK-like” sound. In one or more embodiments, the tern safety mechanism is interchangeable with the terms “locking and unlocking mechanism”.

As used herein the term “fluid communication” refers to two vessels in which substances may pass therethrough either directly or indirectly. The fluid communication may occur via a fluid passageway that allows for the flow/transfer of a medical substance. Optionally, the vessels and/or ports of the systems are provided with seals/covers and those seals/cover become open upon piercing by a piercing member or breaking/rupturing thereof. Optionally, the vessels and/or ports of the systems are provided with seals/covers and those seals/covers are opened upon uncovering or removing thereof by a user. Optionally, one or more of the seals/covers disclosed herein are fragile, allowing piercing thereof with a sharp instrument (i.e., a piercing member) or by the application of a sufficient force to break or rupture the seals/covers. The piercing member may optionally be a needle, for example, a hollowed needle, but other sharp elements are contemplated. In one or more embodiments, the seals/covers may be peelable.

The herein systems and connectors may include one or more valves that restrict fluid flow or passageway. For example, one or more valves may be located at the opening of one or more vessels. In a further exemplary embodiment one or more valves may be disposed at the aperture(s) of the vessels or at a location within a port or vessel body of the vessels. In one or more embodiments, a valve, optionally a one-way valve, may be disposed on and/or within a housing. In one or more embodiments, a valve, optionally a one-way valve, may be disposed on and/or within at least one of a first compartment and a second compartment of a housing. The valves are generally provided to seal and prevent fluid flow prior to any engagement of vessels and/or external displacement of displaceable plates. In an exemplary embodiment, when valves are provided to seal an aperture that provides for the external displacement of displaceable plates, the valves may establish resealing of the apertures at about the time or following the external displacement of the displaceable plates, preventing any leakage of fluids from the engagement between the vessels. In certain embodiments, the resealing occurs at about the time or prior to the external displacement. In certain embodiments, the valves that provide for the passage and external displacement of the displaceable plates may be made of a rubber material, an elastomeric material, a plastic material, and combinations thereof. In an embodiment of the invention, the valve or aperture providing for the passage and external displacement of at least one or two displaceable plates maintains a hermetic seal between at least two vessels or at least two devices or between a vessel and a device or compartment thereof by a force of the engagement. In some embodiments the force of engagement that maintains the hermetic seal between two vessels or devices or vessel-device during the external displacement of the displaceable plates is provided by an engagement mechanism selected from a thread, a luer, a ratchet teeth mechanism, a snap-on mechanism, and combinations thereof. In certain embodiments, the aperture, sealed aperture, or valve providing for the passage and external displacement of at least one or at least two displaceable plates reseal prior to the displaceable plates fully displacing from the first and/or second vessel or device. In some embodiments, the displacement of the displaceable plates occurs in the space between two ratchet teeth. In certain embodiments, the displacement of the displaceable plates occurs between threads of a thread/luer engagement mechanism. In certain embodiments, the valves may prevent backflow of fluids. In some embodiments, the valves may be one-way valves.

In an embodiment of the invention, the herein disclosed devices, systems and methods allow fluid communication in contaminant- free, or substantially contaminant-free manner. For example, between three or more, four or more, five or more or six or more vessels. In an embodiment of the invention, the herein disclosed devices, systems and methods allow fluid communication between three, four, five, six, or seven vessels. For example, the devices, systems, and methods of the invention allow fluid communication between two containers, between two bags, between a syringe and a bag, between a connector and a bag, between a vial and a bag, between a vial and a bottle, between a syringe and a vial or between a syringe and a connector.

A further purpose of this invention is to provide a displaceable plate that reduces the presence of non-purified air and/or air particles. This invention focuses on displaceable plates that are substantially reduced of contaminants and ambient air particles and/or entirely contaminant free and entirely free of ambient air particles.

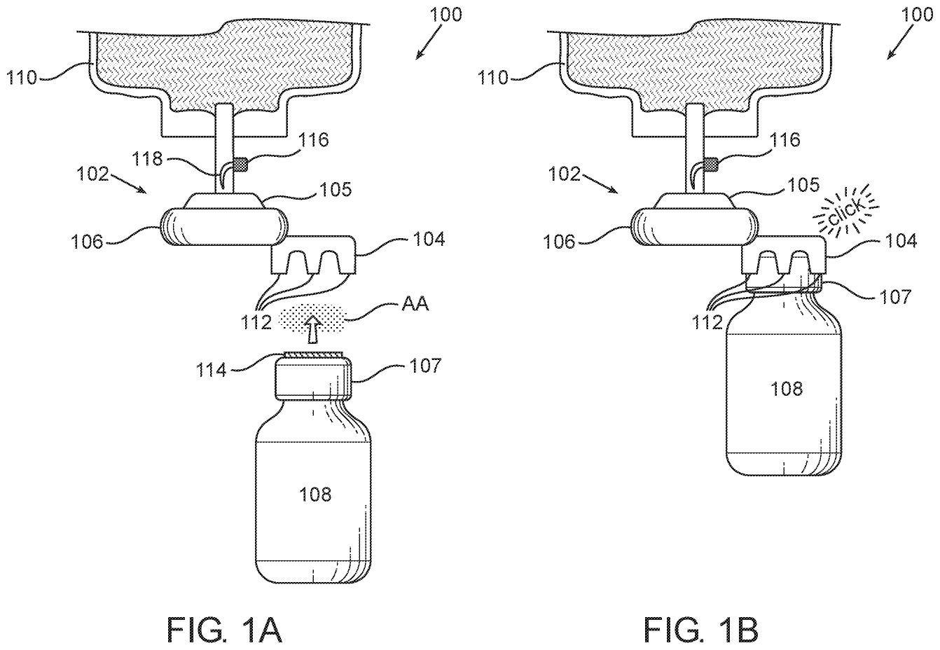

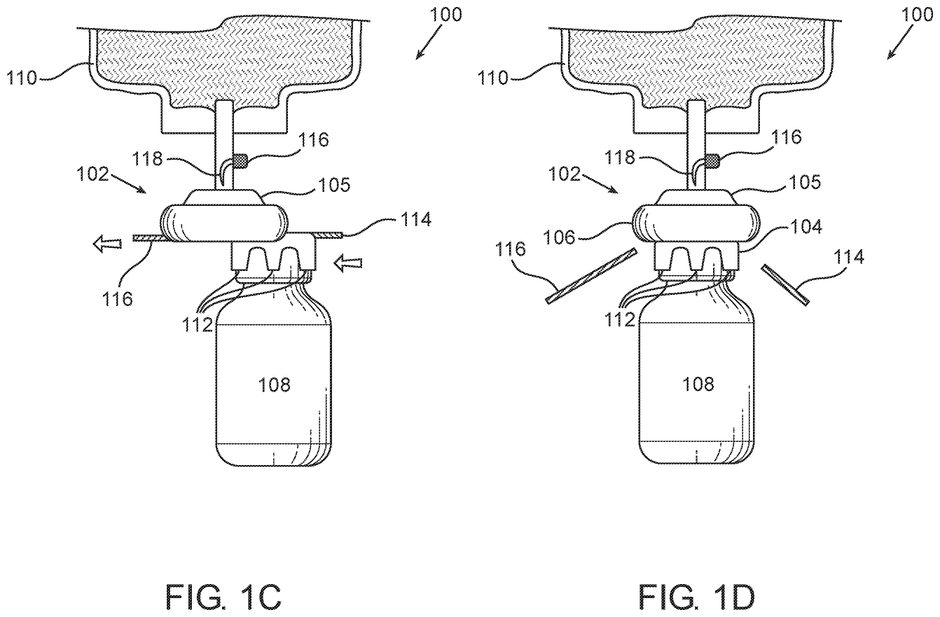

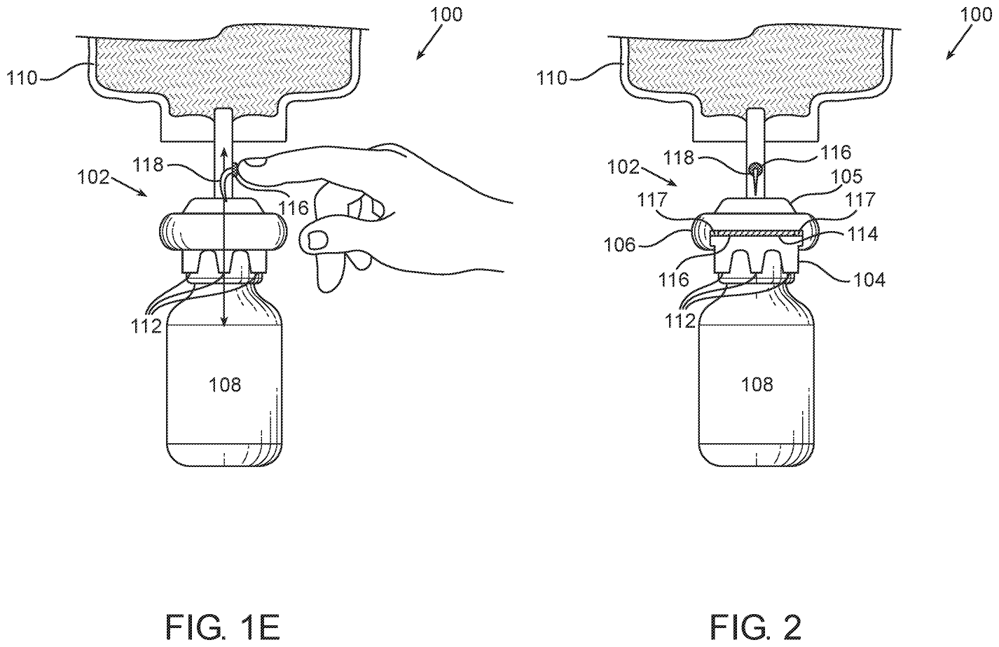

Referring now to the drawings, FIGS. 1A-1E and FIG. 2 illustrate an exemplary decontamination system 100 for the engagement of one or more vessels. System 100 includes a housing 102 with a first compartment 104 and a second compartment 106. First and second compartments 104 and 106 allow an engagement with a medical vessel. For example, first compartment 104 is engageable to first vessel 108 via port 107 and second compartment 106 is provided when connected to second vessel 110 via port 105. The first and second compartments 104 and 106, respectively, each include a bottom opening (not shown) to allow the engagement with a vessel. An engagement mechanism in the form of ratchet teeth 112 constitutes part of first compartment 104 and allows a secure engagement between first compartment 104 and first vessel 108. Each of first and second vessels 108 and 110 include a displaceable plate 114 and 116, respectively which covers a sterile aperture (not shown) of the vessels. Optionally, the displaceable plate is disposed in a slot within the port of the vessel. The displaceable plates 114 and 116 are configured to externally displace at the time, or about the time of movement of first vessel 108 within housing 102. In one or more embodiments, the displacement of the displaceable plates 114 and 116 occurs at the time of, about the time of, in a close proximity to, or consecutively to the engagement between the vessels 108 and 110. In one or more embodiments, the displacement of the displaceable plates 114 and 116 occurs at the time of, about the time of, in a close proximity to, or consecutively to the movement of vessel 108 within housing 102. FIG. 1A illustrates the system 100 when vessel 108 is spaced apart or disconnected from the system 100. Vessel 110 is attached to second compartment 106, optionally integrally attached thereto. Nevertheless, second vessel 110 may be also provided when disconnected from the system 100. As shown, ambient air AA particles surround the interspace between vessel 108 and system 100. At FIG. 1B, vessel 108 engages first compartment 104 via the ratchet teeth mechanism 112 which may connect complementary teeth on vessel 108 (not shown). Ratchet teeth 112 are configured to grasp/clasp port 107 of vessel 108. A sound may be emitted, optionally a click sound is heard at the time of, or about the time of engagement between the first vessel 108 and compartment 104. Optionally, AA particles are entrapped between the engagement of vessel 108 and compartment 104. Vessel 108 is moveable (FIG. 1C), optionally via a sliding motion within a rail of housing 102 and can move from a first position in FIG. 1B to a second position in FIGS. 1D, 1E and 2. At the time of, or about the time of movement, the displaceable plates 114 and 116 displace externally until fully (or partially) removed from the apertures of vessels 108 and 110, respectively. The apertures of the one or both vessels 108 and 110 may optionally further comprise a seal (not shown) which may be disposed below the displaceable plates 114 and/or 116. To allow a fluid communication, the system may thus further include a piercing member 118 which may be operable externally by a user via piercing member actuator 116. A user may press/push actuator 116, thereby piercing the one or more seals, providing the fluid passageway between the vessels 108 and 110 (FIG. 1E). Piercing member 118 may be a needle, optionally a hollowed needle. Displaceable plate 114 may be attached to vessel 108 (and/or port 107) via a frangible seal that is broken upon (at about the time/at the time of) movement of vessel 108 from the first position to the second position. Displaceable plate 116 may be attached to vessel 110 (and/or port 105) via a frangible seal that is broken upon (at about the time/at the time of) movement of vessel 108 from the first position to the second position.

In FIG. 2, the displaceable plate 114 is shown when entrapped between the first compartment 104 and the second compartment 106 and prior to the external displacement. A sliding mechanism, such as a rail 117 between the compartments may be used for the displacement of the displaceable plates and/or movement of the first vessel 108 along the housing 102. The rail 117 may be a part (or integral part) of port 105. The rail may form a unitary structure with port 105. Optionally, the first displaceable plate 114 of first vessel 108 pushes the second displaceable plate of second vessel 110 and thereby allows the external displacement of the first and/or second plates 114 and 116, respectively. Optionally, displaceable plate 116 is concealed within second compartment 106, thereby, maintaining sterility of displaceable plate 116 and of vessel 110 (not shown).

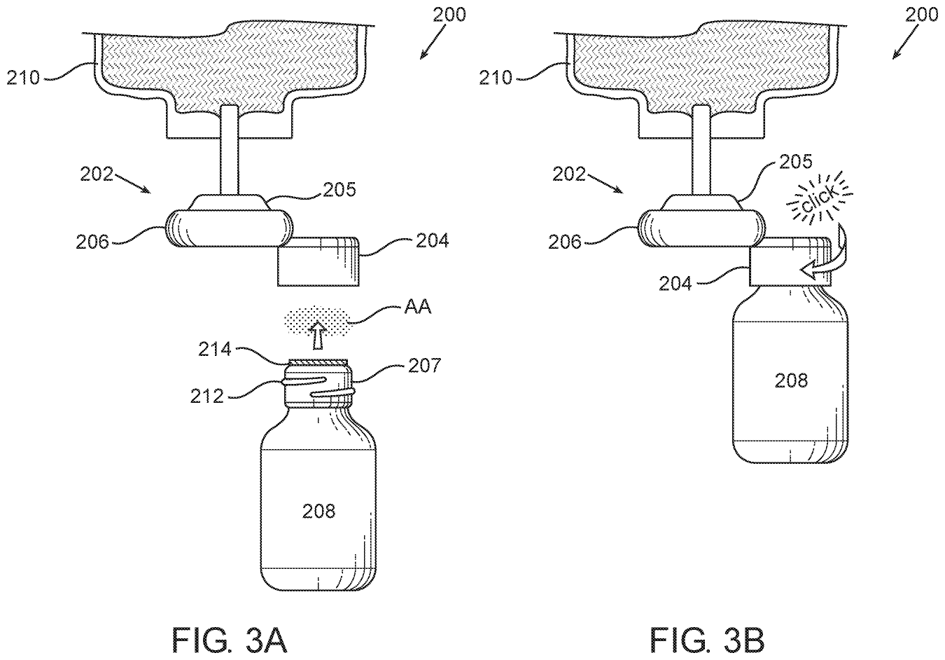

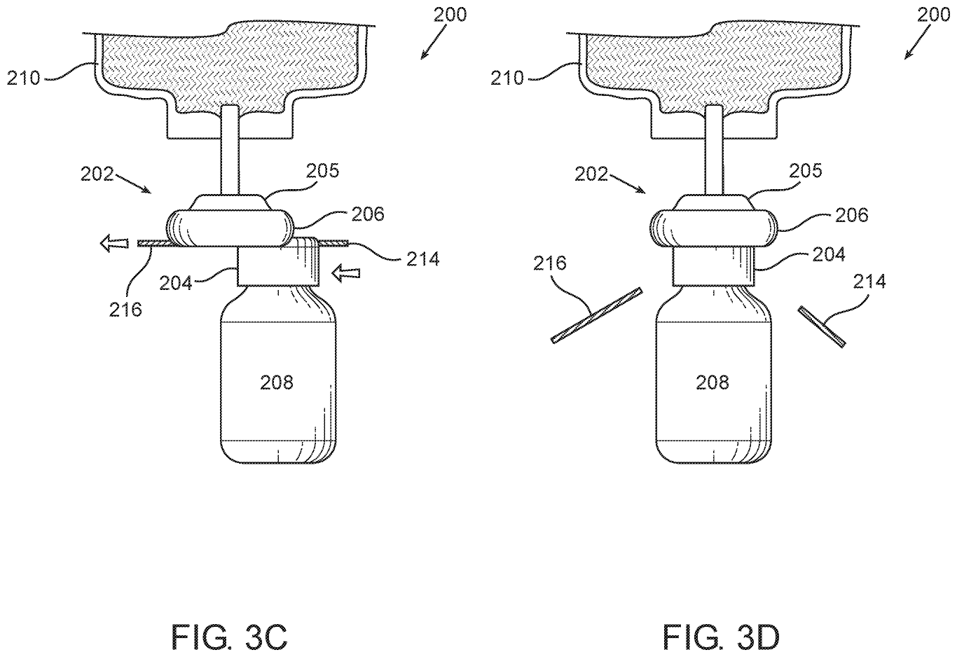

FIGS. 3A-3D illustrate yet another exemplary system 200. Similarly to system 100, system 200 includes a housing 202 with a first compartment 204 for engaging a first vessel 208 and a second compartment 206 for engaging a second vessel 210. Here, the engagement mechanism includes a thread 212 on port 207 of vessel 208 which can cooperate with complementary thread within first compartment 204 of vessel housing 202 (not shown). The engagement by threading is optionally encompassed by a sound, optionally, a click sound, that is emitted at the time of, or about the time of engagement between the vessel 208 and first compartment 204. Second compartment 206 may contain a displaceable plate 216 disposed within the second compartment 206. First compartment 204 and/or second compartment 206 may contain a rail mechanism (not shown) disposed within compartment 204 and/or compartment 206. The rail mechanism (not shown) may be configured to provide for the sliding of at least one of displaceable plate 214 and displaceable plate 216. At least one of displaceable plate 214 and 216 may be disposed between, optionally on, the rail mechanism. When the vessel 208 moves from first position (FIG. 3B) to second position (FIG. 3D), a displacement of the displaceable plates 214 and 216 occurs, allowing a fluid communication and sterile connection between vessels 208 and 210. At FIG. 3D vessel 208 is shown when in a vertical alignment with vessel 210. Displaceable plate 214 which has been exposed to ambient air AA contaminants is displaced/removed from vessel 208 thus decontaminating a surface of vessel 208 resulting in a decontaminated and/or sterile surface of vessel 208. The bottom portion (not shown) of second compartment 206 may optionally be sealed/covered by a seal or a cover. Optionally, the aperture of vessel 208 disposed under displaceable plate 214 may be covered by a seal and/or optionally the aperture of vessel 210 disposed under displaceable plate 216 may be covered by a seal. The seal may be a frangible seal, such as a film, a thin film, a metal alloy film, a plastic film, and combinations thereof that may be pierced/broken by a piercing member and/or application of pressure to the seal. FIG. 3C and FIG. 3D shows how the movement of vessel 208 from the first position to the second position displaces displaceable plates 214 and 216. Optionally, contaminated (non-sterile due to exposure to ambient air AA) displaceable plate 214 may be displaced from vessel 208 without the displacement of displaceable plate 216 (which may be concealed within sterile second compartment 206).

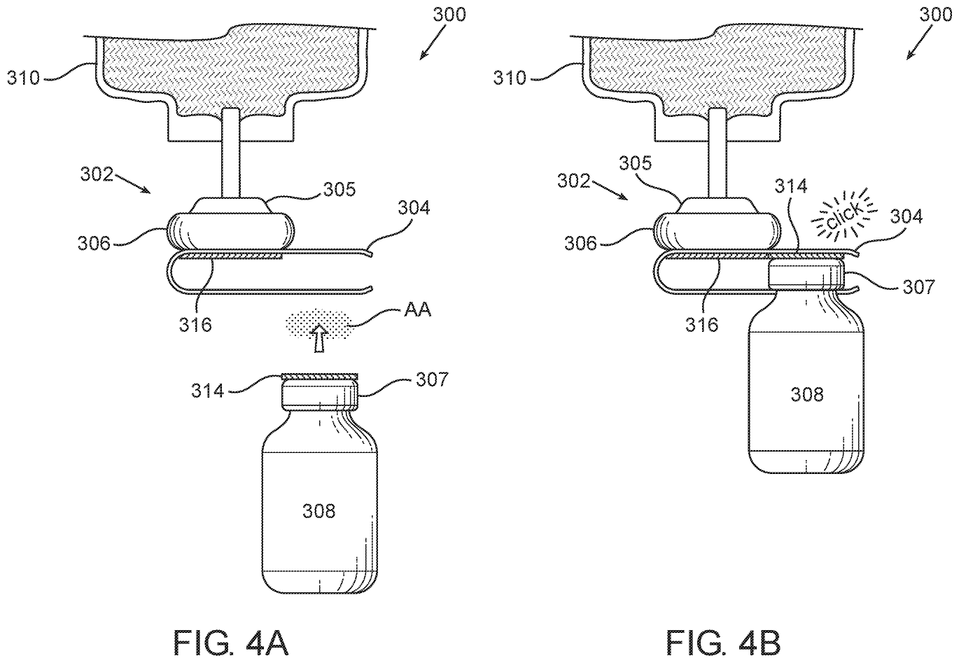

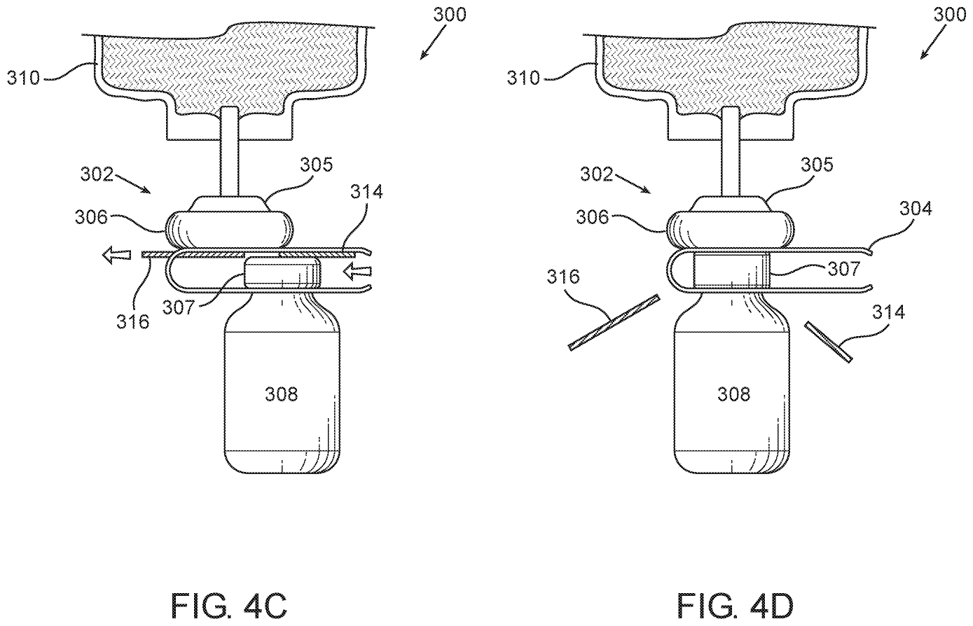

FIGS. 4A-4D illustrate yet another exemplary system 300 which similarly to the preceding figures includes a housing 302, with a first compartment 304 and a second compartment 306. First compartment 304 has a clamping rail structure and mechanism, allowing the sliding of vessel 308 from a first position (FIG. 4B) to a second position (FIG. 4D). Optionally, alternatively or additionally, the clamping rail structure allows the displacement of the displaceable plate(s) 314 and/or 316. The clamping rail 304 includes an opening (not shown) via which port 307 of first vessel 308 may engage with housing 302. Port 307 of vessel 308 may engage the clamping rail from the bottom opening (not shown), or optionally from the side opening. The clamping rail 304 further includes a second opening which allows accommodating therein displaceable plate 316 of vessel 310. At FIG. 4A, the vessels 308 and 310 are shown when spaced apart. At FIG. 4B, the vessel 308 disposed in first position, wherein the vessel engages housing 302 via first compartment and attach the clamping rail 304. A click (or any other audible) sound may optionally encompass the engagement with the housing 302. The displaceable plate 314 may abut a side wall of displaceable plate 316 and disposed in a horizontal alignment with displaceable plate 316. At FIG. 4C, port 307 of first vessel 308 slides within the clamping rail 304 to align vertically with vessel 310. The displaceable plates 314 and 316 displace externally and the engagement between the vessels 308 and 310 is allowed via the sterile aperture of the vessels (not shown). Once aligned, a fluid communication may be formed between the vessels 308 and 310. Displaceable plate 314 and/or 316 may be attached to respective vessel 308 and/or 310 via a frangible seal. The force of a user's hand when moving vessel 308 from the first position to the second position may provide the necessary force to displace at least one of displaceable plates 314 and 316. The force of a user's hand when moving vessel 308 from the first position to the second position may provide the necessary force to break the frangible seal that attaches displaceable plate 314 to vessel 308 and/or displaceable plate 316 to vessel 310.

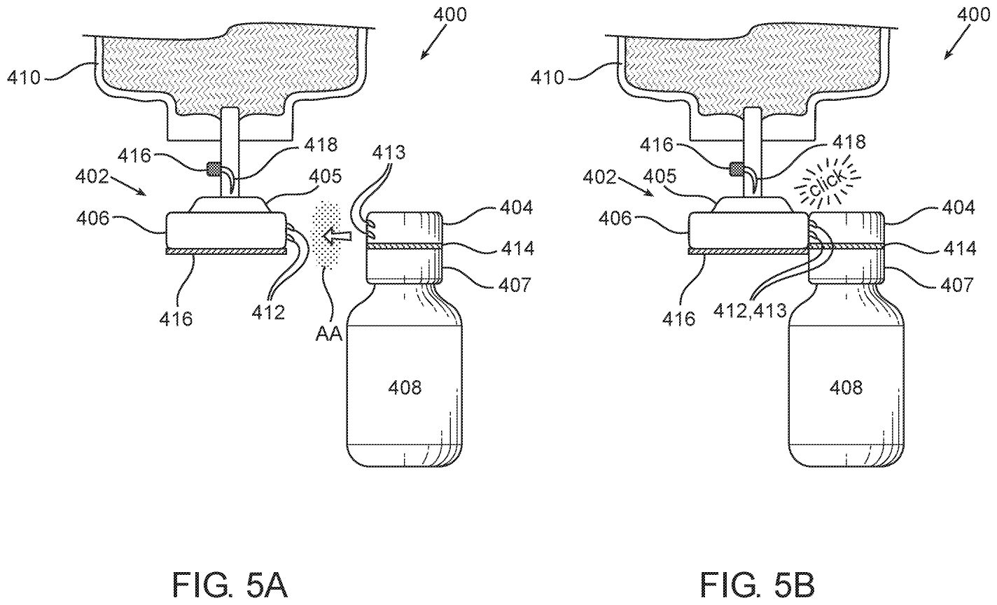

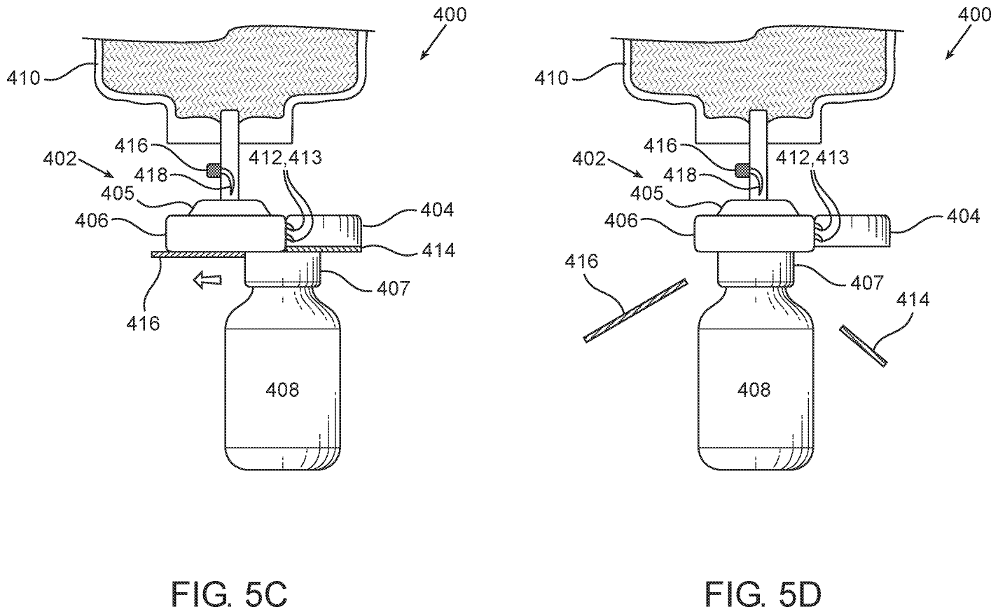

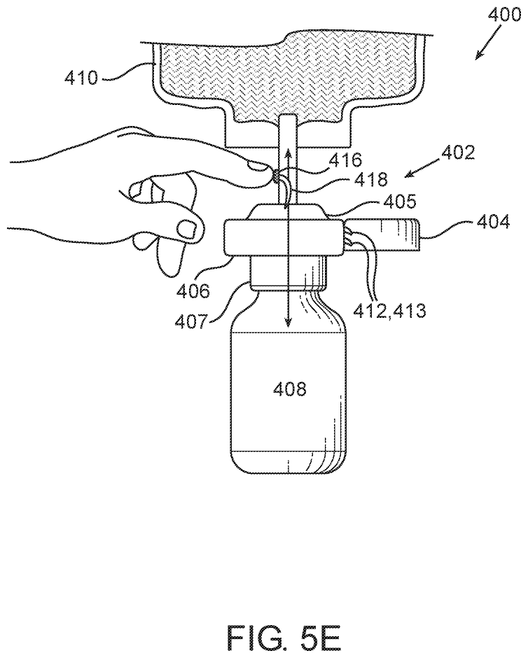

FIGS. 5A-5E illustrate yet another exemplary system 400 which includes a housing 402 with first compartment 404 and a second compartment 406. Here, the first compartment 404 is provided when connected to first vessel 408, optionally on top of first displaceable plate 414 and thereby attached to port 407. Optionally, displaceable plate 414 may be directly attached to vessel 408 without the presence of port 407. In accordance with this embodiment displace plate 414 may abut vessel 408, displaceable plate 414 may abut a wall of vessel 408, displaceable plate 414 may be surface mounted to vessel 408, and/or displaceable plate 414 may be flush mounted to vessel 408. An engagement mechanism in the form of teeth 412 and dents 413 allows the secure connection between the compartments 404 and 406. At the time of engagement of first compartment 404 to second compartment 406 (FIG. 5B), a click sound is optionally emitted alerting the user that the engagement step has been established. The displaceable plates 414 and 416 disposed substantially in a horizontal alignment after the compartments' attachment. In FIG. 5B (contaminated) ambient air AA is entrapped between first compartment 404 and second compartment 406. In FIG. 5B (contaminated) air AA is entrapped between displaceable plate 414 and displaceable plate 416. Vessel 408 can then slide along housing 402 and thereby allow the external displacement of displaceable plates 414 and 416 (FIGS. 5C and 5D). Displaceable plates 414 and 416 are shown completely detaching (falling off) of their respective vessels 408 and 410. However, in some embodiments, after displacement of at least one of displaceable plates 414 and 416 from at least one of vessels 408 and 410 at least one of displaceable plates 414 and 416 may remain attached to at least one of first compartment 404 and second compartment 406. At FIG. 5E, vessel 408 is at second position in housing 402 and a fluid passageway may be formed between the vessels 408 and 410 in a substantially sterile manner, in a substantially decontaminated manner, and/or a substantially air-free manner.

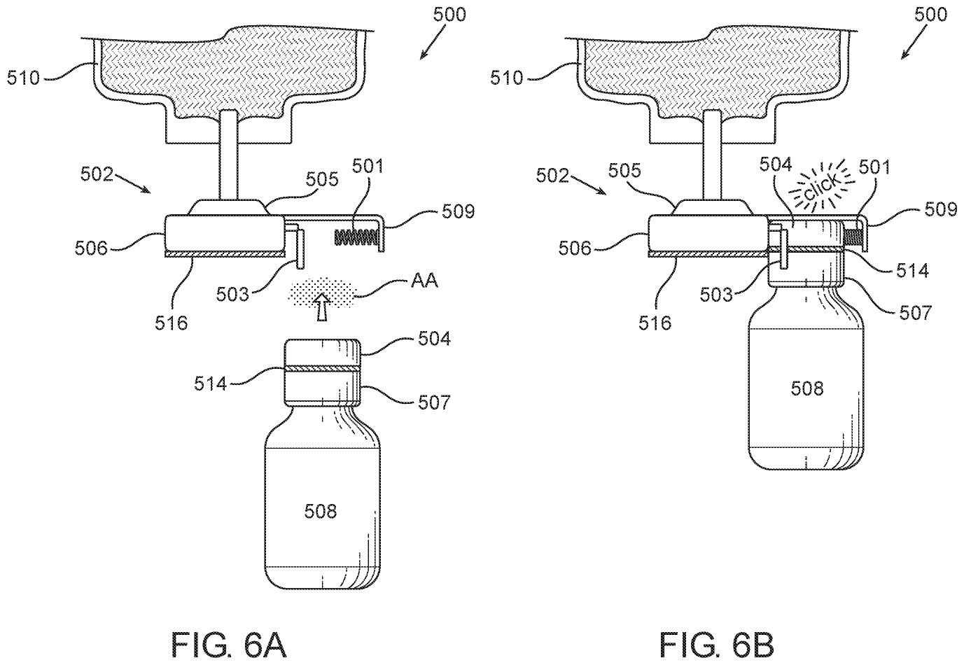

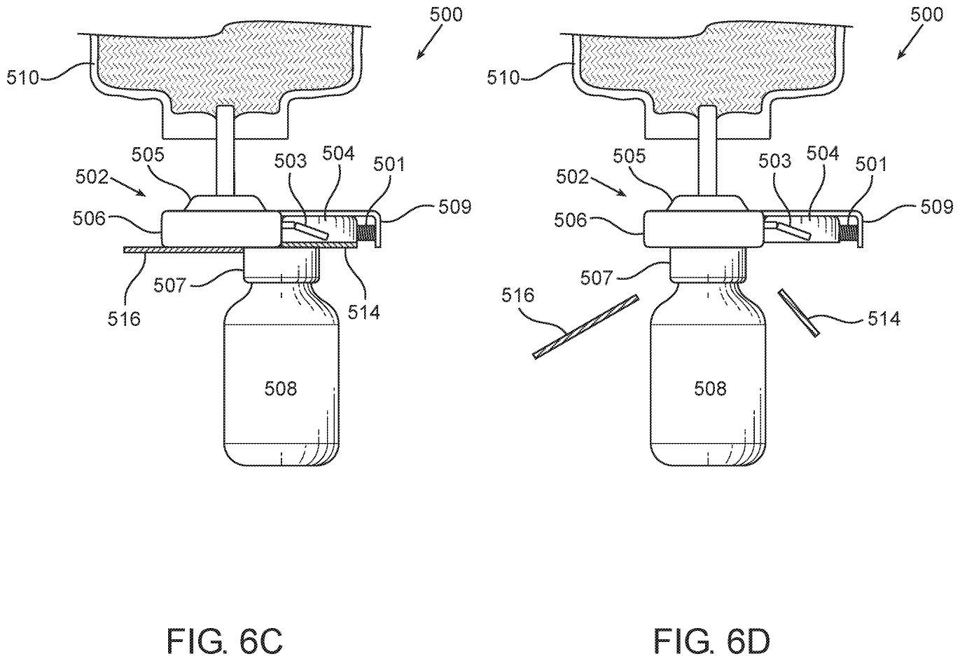

Yet a further exemplary decontamination system 500 is shown in FIGS. 6A-6D. Here the system 500 includes a housing 502 with a second compartment 506 and a rigid frame element 509 for accommodating therein first compartment 504. A pressing mechanism having a spring 501 within first compartment 504 allows a tight connection between the compartments 504 and 506. A locking mechanism 503 is further optionally provided to lock together first compartment 504 and second compartment 506. At FIG. 6A, vessel 508 is shown when a sterile aperture thereof is covered by a displaceable plate 514 and first compartment 504 is on top of the displaceable plate 514. AA surround the vessel 508 and housing 502. At FIG. 6B, vessel 508 is attached to housing 502. Spring 501 is contracted and presses the first compartment 504 against a side wall of second compartment 506. The locking mechanism 503 locks together first compartment 504 and second compartment 506. At FIG. 6C, vessel 508 detaches from an engagement with first compartment 504 and moves within and/or along housing 502. The unlocking mechanism 503 allows the movement and unlocks the vessel 508 from the engagement with first compartment 504. At FIG. 6D the displaceable plates 514 and 516 fall off or displaces externally and vessel 508 is positioned in the second position and aligned vertically with vessel 510. A fluid passageway may be now formed between the vessels.

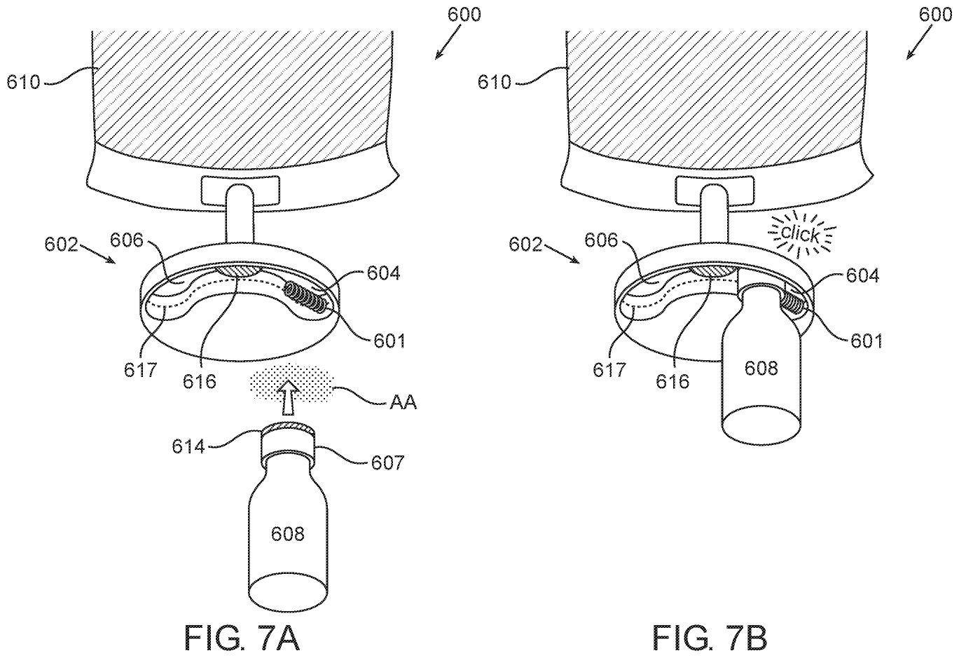

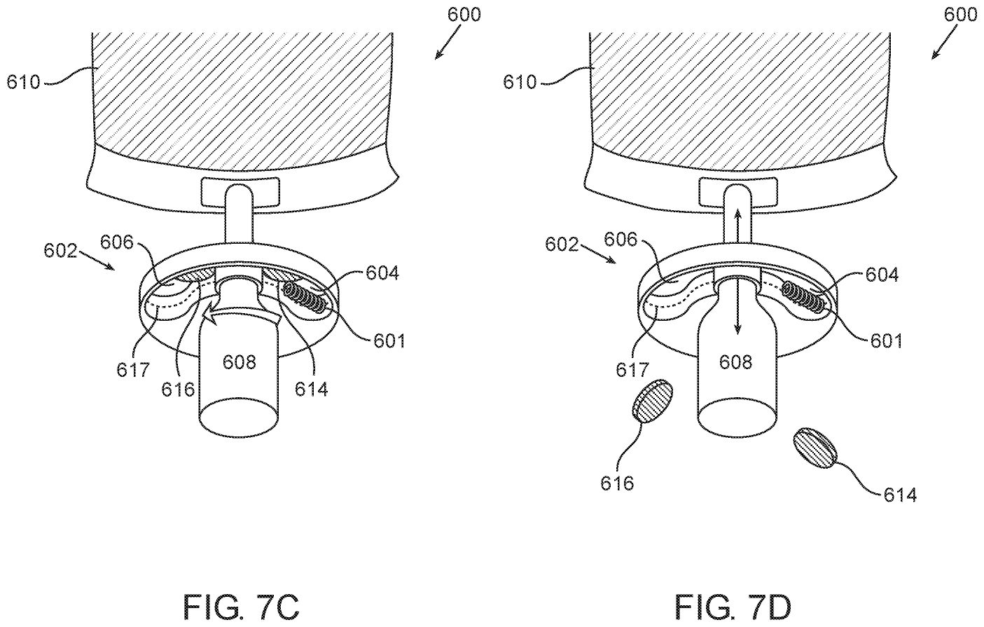

FIGS. 7A-7D illustrate yet another exemplary system 600. Here the housing 602 is rounded in shape and includes first compartment 604 and second compartment 606 which are integral parts of the housing 602. Each of the vessels 608 and 610 includes a substantially sterile aperture which is covered by a displaceable plate 614 and 616, respectively. A pressing mechanism with spring 601 is disposed adjacent or as part of first compartment 604 and tightly presses port 607 of vessel 608 within the housing 602 and against displaceable plate 616 as shown in FIG. 7B. A click sound (or any audible sound) may be emitted when the vessel 608 engages housing 602 (FIG. 6B). First vessel 608 (or port 607 of vessel 608) may slide within the housing 602, optionally via a sliding mechanism having a rail 617 and move to second compartment 606. The movement of vessel 608 may effect the displacement of plates 614 and 616, thereby allowing a fluid communication between vessel 610 and 608 when the vessels are vertically aligned. Housing 602 is shown with an open bottom, however the bottom, in some embodiments, may be closed/sealed by a cover/seal. In some embodiments, first compartment 604 may be covered/sealed by a cover/seal. In some embodiments, the second compartment 606 may be covered/sealed by a cover/seal. In some embodiments both first compartment 604 and second compartment 606 may be covered/sealed by a cover/seal. FIG. 7D shows displaceable plates 614 and 616 falling off (completely detaching from) housing 602. However, in some embodiments, at least one of displaceable plates 614 and 616 may be displaced within housing 602 wherein the bottom of housing 602 is covered by a cover/seal in which case at least one of displaceable plates 614 and 616 will be displaced from respective vessel 608 and 610 and wherein at least one of displaceable plates 614 and 616 will remain inside housing 602.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for a decontaminated engagement of vessels, the system comprises:
a housing comprising a first compartment connectable to a first vessel's port; and a second compartment connectable to a second vessel's port,
wherein the first and second compartments are engageable with each or integrally connected to each other, and wherein the first vessel is moveable within the housing from a first position wherein the vessel is connected to the first compartment, to a second position wherein the vessel is connected to the second compartment, wherein a substantially sterile aperture of the first vessel and/or second vessel is covered by a displaceable plate, and wherein prior to or at about the time of connection of the first vessel in the second position, the displaceable plate displaces from the aperture, allowing an substantially decontaminated engagement and fluid passageway between the first and second vessels.

2. The system of claim 1, wherein the first compartment is provided when attached to a port of the first vessel.

3. The system of claim 1, wherein the first and second compartments are engageable with each other via an engagement mechanism.

4. The system of claim 3, wherein the engagement mechanism is selected from a thread, a luer, an adhesive, a clamping rail, a teeth and complementary dents, a clamping arm and a ratchet teeth mechanism.

5. The system of claim 1, wherein the first and second compartments are horizontally engageable with each allowing an axial movement of the first vessel within the housing.

6. The system of claim 1, wherein the second position of the first vessel comprises a substantially vertical alignment between the first and second vessels.

7. The system of claim 1, wherein the displaceable plate displaces externally.

8. The system of claim 1, wherein the displaceable plate is a film, a plastic, a metal alloy, or a combination thereof.

9. The system of claim 1, wherein the movement of the first vessel within the housing is via a sliding mechanism.

10. The system of claim 9, wherein the sliding mechanism includes a rail.

11. The system of claim 9, wherein the sliding mechanism disposed on the first vessel, second vessel, first compartment and/or second compartment.

12. The system of claim 1, wherein the first vessel and the second vessel each include a displaceable plate and wherein the displaceable plates externally displace about the same time, or consecutively.

13. The system of claim 1, wherein the displaceable plate displaces from the aperture at about the time of movement of the first vessel within the housing.

14. The system of claim 1, wherein the system allows a substantially air particle-free engagement between the first vessel and the second vessel, wherein air particles are entrapped between the first displaceable plate and the second displaceable plate.

15. The system of claim 1, further comprising a piercing member on said first vessel and/or said second vessel configured to pierce a cover or seal on said aperture, thereby allowing a fluid passageway between the vessels.

16. The system of claim 1, wherein an audible sound is emitted at or about the time of engagement between the first compartment and the second compartment, displacement of the displaceable plate, engagement between the first and second vessels, movement of the first vessel in the housing and/or formation of a fluid passageway between the vessels.

17. The system of claim 1, further comprising a locking-unlocking mechanism between the first compartment and the second compartment, the locking mechanism configured to lock the compartments to each other.

18. The system of claim 1, further comprising a pressing mechanism on at least one of said first compartment and second compartment, the pressing mechanism configured to allow a tight connection between the compartments.

19. The system of claim 18, wherein the pressing mechanism includes a spring that presses the first compartment against a wall of the second compartment.
